(19) United States
(12) Patent Application Publication
(10) Pub. No.: US 2024/0180720 A1
(43) Pub. Date: Jun. 6, 2024

(54) ANCHORING SYSTEM FOR UPPER LIMB BODY-POWERED PROSTHETICS HARNESS

(71) Applicant: Iowa State University Research Foundation, Inc., Ames, IA (US)

(72) Inventors: Bahar Hashemian Esfahani, Ames, IA (US); Rachel Jean Eike, Ames, IA (US)

(21) Appl. No.: 18/525,226

(22) Filed: Nov. 30, 2023

Related U.S. Application Data
(60) Provisional application No. 63/386,216, filed on Dec. 6, 2022.

(51) Int. Cl. A61F 2/54; A61F 2/68; A61F 2/78

(52) U.S. Cl. CPC A61F 2/54; A61F 2002/6872; A61F 2002/7862

(57) ABSTRACT

A bracket is used as part of a harness, such as those used with prosthetic devices for upper limb loss. The bracket is part of an anchoring system that can be used with a garment/harness, such as a figure of 9 style harness. The bracket includes features that include improved material, which are less dense and stronger than that currently used. In addition, the material allows for better sensorial comfort by the user. The design also improves the pressure due to the improved center of gravity placement of the bracket on the user, while also providing an easy to attach feature and ambidextrous ability.

### CROSS-REFERENCE TO RELATED APPLICATIONS

This application claims priority under 35 U.S.C. § 119 to provisional patent application U.S. Ser. No. 63/386,216, filed Dec. 6, 2022. The provisional patent application is herein incorporated by reference in its entirety, including without limitation, the specification, claims, and abstract, as well as any figures, tables, appendices, or drawings thereof.

### TECHNICAL FIELD

The present disclosure relates generally to prosthetic devices. More particularly, but not exclusively, the disclosure relates to brackets, systems, anchoring systems, and harnesses for use with prosthetic devices.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

There are approximately 2.1 million people living with limb loss in the United States, and that number is expected to double by 2050. Losing an upper limb has a significant effect on one's life since hands and arms are used for considerable daily activities. As a result of challenges experienced with limb loss, prosthetic devices have been developed to serve as a substitute limb. Such prosthetic devices require individualized fabricated and fitted prostheses. Body-powered prosthesis is the most commonly used prosthetic device for people with upper limb amputation, especially ‘Figure of 9’-style harnesses for unilateral transradial prostheses. However, users have reported being unhappy with the appearance, pain, discomfort, and dissatisfaction of the prosthesis system.

Therefore, there is a need in the art to address the needs of amputees using the ‘Figure of 9’ harnesses and to develop an alternative design solution to build upon or replace the ‘Figure of 9’ harness.

Since the early 1990s, some additional harnesses for upper limb amputees and/or prosthetic devices have been developed. These include U.S. Pat. No. 8,323,355 to LaTour; PCT Publication No. 2010120404 to Altobelli et al.; and U.S. Pat. No. 9,737,374 to Doyle, all of which are hereby incorporated by reference in their entirety. These harnessing options vary in materials used as well as how they interact or adhere to the body. The '355 patent to LaTour also includes an anchoring system that does not wrap around the body like most harness options, but rather uses medical-grade adhesives to place an anchor point on the body for the control cable to connect with to allow for actuation of the terminal device. All these disclosures referenced use metals in their system attached to the body.

Many concerns with the figure of 9 harness include feelings of pain and pressure on the unamputated side, feeling pain and redness around the axilla area because of friction against the skin while using the harness. There have also been complaints about having pain around the neck and shoulder and also about the harnessing system which is not removable and replaceable.

Therefore, there is also a need in the art looking at the needs of amputees without upper extremity limbs using the Figure of 9 harnesses, to improve upon and/or address the limitations included in the cited patents and publications.

### SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage include a bracket for use with a harness and/or prosthetic device that uses a less dense material. For example, polyethylene terephthalate glycol (PETG) is less dense than iron, which has previously been used.

It is still yet a further object, feature, and/or advantage to improve the sensorial comfort of a bracket used with a harness and/or prosthetic device.

It is another object, feature, and/or advantage to provide a harness and/or prosthetic device that is body-friendly and washable and/or launderable.

It is yet another object, feature, and/or advantage to centralize a pressure load area for a bracket or harness for a prosthetic device to locate the pressure at a center of gravity of the bracket and/or body of the user.

It is still a further object, feature, and/or advantage to provide a symmetrical bracket for use with a harness and/or prosthetic device that allows for ambidextrous use.

The bracket, harness, and/or combination disclosed herein can be used in a wide variety of applications. For example, the bracket and/or harness incorporating the same could be used with a number of prosthetic devices, including a cord operated end effector. The bracket could be used with a number of harness types as well.

It is preferred the apparatus be safe, cost effective, and durable. For example, the bracket and/or harness disclosed can be adapted to resist excessive heat, static buildup, corrosion, and/or mechanical failures (e.g., cracking, crumbling, shearing, creeping) due to excessive impacts and/or prolonged exposure to tensile and/or compressive forces acting on the apparatus. The use of PETG also allows for washing of the bracket without the worry of rust or other corrosion. For example, the harness and bracket could be washed by hand or in net bags placed in a home laundering machine.

At least one embodiment disclosed herein comprises a distinct aesthetic appearance. Ornamental aspects included in such an embodiment can help capture a consumer's attention and/or identify a source of origin of a product being sold. Said ornamental aspects will not impede functionality of the bracket.

Methods can be practiced which facilitate use, manufacture, assembly, maintenance, and repair of a bracket which accomplish some or all of the previously stated objectives.

The bracket can be incorporated into systems, harnesses, and/or other prosthetics which accomplish some or all of the previously stated objectives.

According to some aspects and/or embodiments of the present disclosure, a harness system comprises a harness comprising front and rear portions connected by an open upper area, said front and rear portions extending generally downward at central locations; and a bracket positioned at the rear portion of the harness, said bracket comprising upwardly V-shaped channels; wherein said bracket positioned on the rear portion of the harness in a manner to position the bracket at a generally center portion of the rear portion.

According to at least some aspects of some embodiments, the harness system further comprises a prosthetic device operatively connected to the bracket.

According to at least some aspects of some embodiments, the prosthetic device comprises a cable attached to one or both of the upwardly V-shaped channels of the bracket.

According to at least some aspects of some embodiments, the prosthetic device further comprises an end effector actuated via the cable.

According to at least some aspects of some embodiments, the bracket comprises polyethylene terephthalate glycol (PETG).

According to at least some aspects of some embodiments, the bracket comprises an irregular hexagon shape.

According to at least some aspects of some embodiments, the bracket is symmetrical.

According to at least some aspects of some embodiments, the upwardly V-shaped channels comprise larger openings at the upper portions of the channels.

According to at least some aspects of some embodiments, the upwardly V-shaped channels are in a golden triangular configuration.

According to at least some aspects of some embodiments, the harness further comprises straps extending from opposite sides of the front and rear portions at lower portions thereof.

According to yet additional aspects of the present disclosure, a bracket for use with a harness system including a prosthetic device includes a bracket body; upwardly V-shaped channels extending through the bracket body, said channels being symmetrical and angled to form a golden triangle; wherein said bracket comprising PETG.

According to at least some aspects of some embodiments, the bracket body is in an irregular hexagon shape.

According to at least some aspects of some embodiments, the bracket body is symmetrical.

According to at least some aspects of some embodiments, the upwardly V-shaped channels comprise larger openings at the upper portions of the channels.

According to at least some aspects of some embodiments, the bracket further comprises a plurality of apertures positioned around the periphery of the bracket body for attachment to a harness.

According to at least some aspects of some embodiments, there are rivets positioned at the plurality of apertures to attach the bracket body to the harness.

According to yet additional aspects of the present disclosure, a harness for use with a prosthetic device includes a harness body comprising front and rear portions connected by an open upper area, said front and rear portions extending generally downward at central locations; a bracket positioned at the rear portion of the harness, said bracket comprising upwardly V-shaped channels; and straps extending from opposite sides of the front and rear portions at lower portions thereof; wherein said bracket positioned on the rear portion of the harness in a manner to position the bracket at a generally center portion of the rear portion.

According to at least some aspects of some embodiments, said straps being length adjustable.

According to at least some aspects of some embodiments, said bracket comprising a symmetrical hexagon shape with inwardly oriented lower sections.

According to at least some aspects of some embodiments, said bracket comprises a plurality of attachment apertures for attaching the bracket to the harness body.

According to at least some aspects of some embodiments, the upwardly V-shaped channels comprise larger openings at the upper portions of the channels.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 is a depiction of a user wearing a harness system including a prosthetic device and including a harness according to aspects and/or embodiments of the present disclosure.

FIG. 2 is another depiction of the user and the harness system showing activation of the prosthetic device.

FIG. 3 is a rear depiction of the user and harness system.

FIG. 4 is a closeup showing a bracket for attaching the prosthetic to the harness for the harness system.

FIG. 5 is an image showing how a prosthetic device can be connected to the bracket of the harness according to aspects and/or embodiments of the present disclosure.

FIG. 6 is a view of a bracket for use with a harness according to aspects and/or embodiments of the present disclosure.

FIG. 7A is another view of the bracket of FIG. 6 showing an overlayed golden triangle.

FIG. 7B is a view of the golden triangle of FIG. 7A.

FIG. 8 is another view of the bracket of FIG. 6.

An artisan of ordinary skill in the art need not view, within isolated figure(s), the near infinite distinct combinations of features described in the following detailed description to facilitate an understanding of the present disclosure.

### DETAILED DESCRIPTION

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

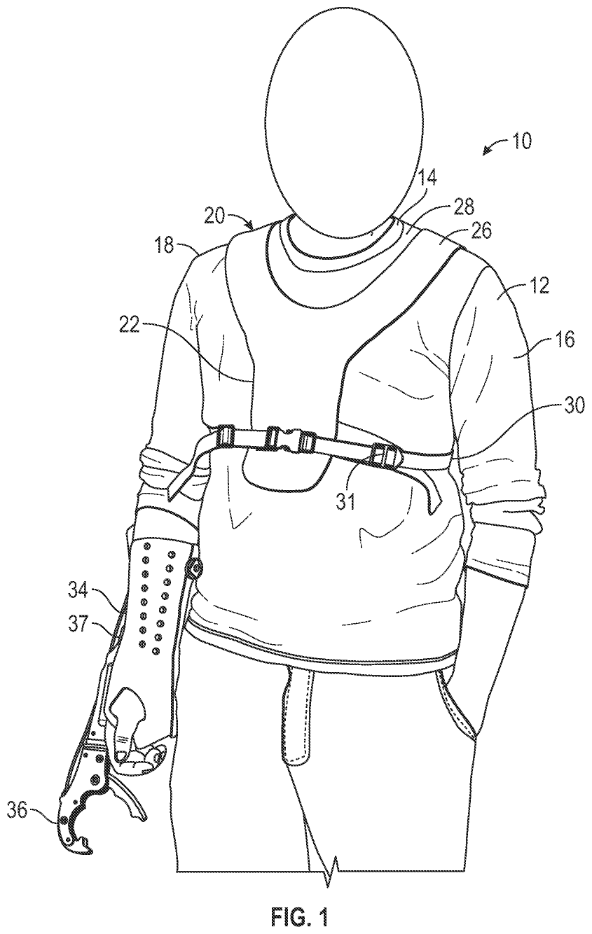

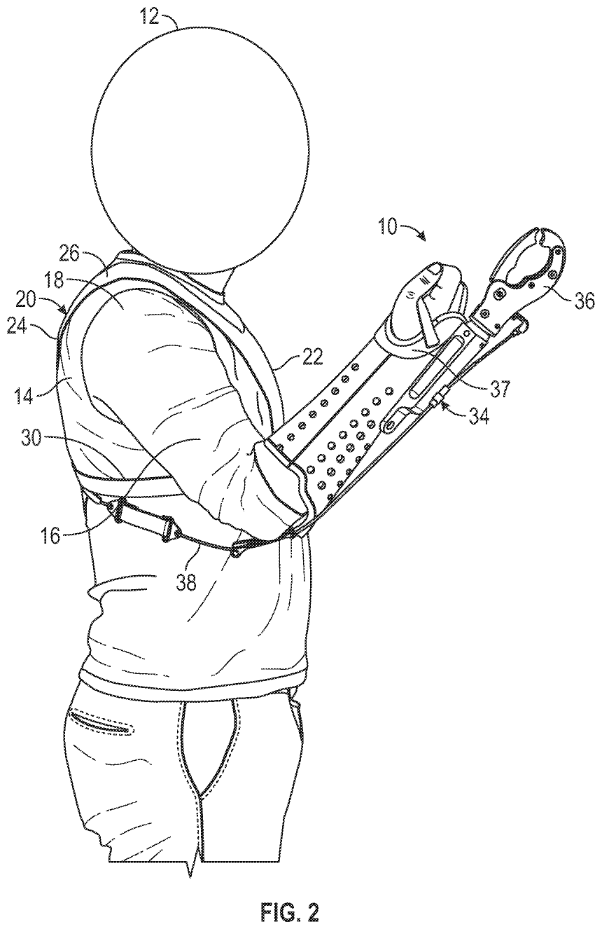

FIGS. 1-4 depict the use of a harness system 10 includes aspects and/or embodiments of the present disclosure. For example, FIGS. 1-3 show the harness system 10 being attached to or on a user 12, who is shown for example purposes. As noted, traditional harnesses utilize anchoring via the Figure of 9 design or attachment to a portion of a user, wherein a prosthetic device, such as the device 34 shown in the figures, is connected.

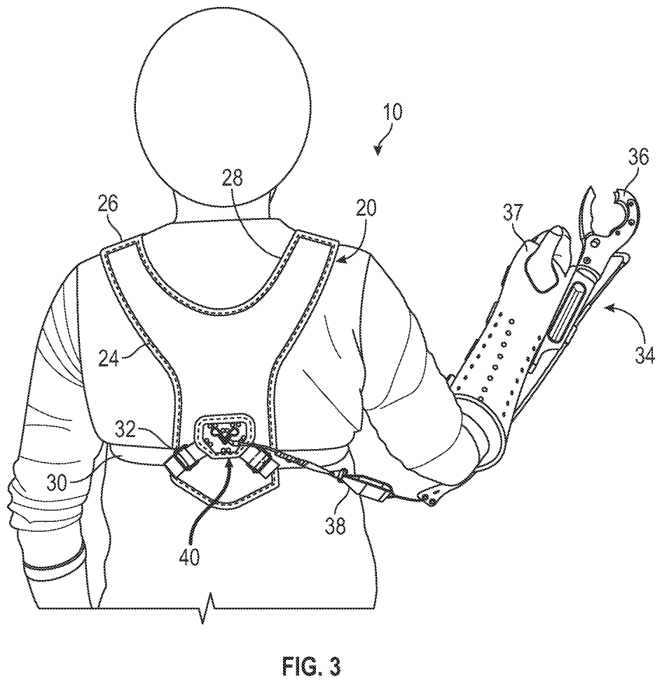

The system 10 shown in the figures is an improvement upon such harnesses, as it provided numerous advantages. As shown, the harness system 10 is used generally with or at the upper body 14 of the user 12, which includes the upper torso, upper limbs 16 (i.e., arms), shoulders 18, and the neck area 19. The system 10 includes a harness 20 including a front portion 22 and a rear portion 24. The front and rear portions 22, 24 are connected by shoulder straps 26 that are designed to go on opposite shoulders, creating an upper open portion 28. This allows the shoulder straps 26 to rest on the shoulders 18 with the front portion 22 extending downwardly to cover a portion of the front of the user 12, such as in a middle portion of their body. Likewise, as shown in FIG. 3, the rear portion 24 extends generally downwardly from the shoulder straps 26 at a midsection of the user's back. The new harness is designed based on body shape on the front and back to provide comfort when user is wearing it.

Advantages of the front portion 22 of the harness include, but are not limited to, keeping the fabric around the neck wide and open to ease of taking on/off and taking the webbing straps down from axilla to the chest to decrease the friction against the skin in the axilla area.

Another feature of the harness 20 was removing the straps 26 from the neck area of the original figure of 9 harness design to a mid-point between the neck and shoulder, such as is shown in the figures. The neckline was also re-designed to have a wider opening/closing for the head to enter to provide easier donning and doffing.

The harness 20 is included in two layers; the outer layer with natural fabric (100% cotton canvas) which absorbs moisture (repelling sweat) rather than repelling it (forcing it to sit on a user's body). The canvas fabric is also more skin-friendly and mitigates dampness as compared to traditional figure of 9 harnesses. The second layer, which is sandwiched between natural fabric is scuba fabric to provide more strength by thickness to carry the weight on the bodice not just one side of the body. Furthermore, as will be understood, the whole harness design (with bracket sewn to it) are able to be easily laundered/washed.

Additional aspects of the harness system 10 include adjustable straps 30 connecting the front and rear portions 22, 24 of the harness 20, which provides additional support for the use of the harness system 10. The adjustable straps 30 are adjustable via members 31. The ends of the adjustable straps 30 are shown to be connected at the rear portion 24 of the harness 20 at connectors 32, which are generally fixed. The straps 30 are connectable to one another at a buckle positioned at the front portion 22 of the harness 20. The harness is designed to take down the straps around the body to address the problem of friction around the axilla area and to bring more comfort that is distributed on both sides of the body.

The harness 20 is easy to use: The buckle is placed on the front portion 22 in which one side of the buckle is attached to the harness and the other strap is free to make donning/doffing easier for single-handed users. Squeezing the plastic construction of the buckle releases it quickly and easily. The buckles and straps address the fitting problem of amputees as well because with just one hand they can make the harness tighter or looser with different body sizes. In other words, with this single adjustable side release buckle in place, they can quickly unhook the harness straps.

Additional aspects include the addition of additional material for female use, such as additional covering at the bust area. This could be sewn or otherwise attached to the should straps, as well as the front and rear portions for additional covering and support.

As shown in the Figures, a prosthetic device 34 may be used as part of the harness system 10. The prosthetic device can be used by someone such as an amputee or someone born with a limb difference or other reason why they may want to add to the abilities of an appendage, such as a hand or arm. The example prosthetic shown in the figures is a using a prosthetic device simulator performing Glenohumeral Flexion, Bi-lateral scapular abduction, and Elbow or Forearm Flexion motions, which are commonly used as baseline movement expectations for prostheses actuation. This is a type of cable-activated elbow. Cable-operated elbows, sometimes called body-powered elbows, use a system of cables connected to a harness, to bend or straighten the elbow. This technology does not require manual positioning as shoulder movements allow for adjustments of the elbow. How the elbow operates within the harness system can be customized, including creating bilateral cable-operated solutions.

In body-powered upper-limb prosthetic applications, the functions of control and suspension are closely interrelated. The prosthesis is suspended on the residual limb by the intimacy of the socket fit and by harness 20. In a well-designed harness the same straps are strategically positioned in relation to the shoulder girdle and/or thorax so that the amputee can control the prosthetic components with a minimum of exertion and body motion. The prosthesis 34 shown is an example of a below-elbow (transradial) control system.

The transradial prosthetic control system is a one-cable or “single-control” system. A stainless steel control cable is firmly attached at its proximal end to the harness 20, such as at a bracket 40 as will be disclosed. Distally, the cable terminates at some type of prehension device, which may also be referred to as an end effector.

Prehension devices, usually referred to simply as “terminal devices,” may be either prosthetic hands with one or more movable fingers or two-fingered devices with a hook-type configuration. With this type of terminal device, the amputee uses shoulder motion on the amputated side to apply tension to the control cable. The cable tension is transmitted to the operating lever or “thumb” of the terminal device and causes one finger of the hook to move away from the other stationary finger. When cable tension is relaxed, the movable finger closes on the stationary finger.

The cable 38 is part of a cover 37, which may cover a portion of the appendage (i.e., arm or hand). The cable 38 can actuate an end effector 36. Cable-operated elbows are usually paired with end effectors 36 in the form of mechanical hands or hooks that can be controlled with an additional, integrated cable system 38. Therefore, it is key to have a good connection between the prehension devices and the harness through the cable, so that there can be easy activation, as well as comfort, for the user.

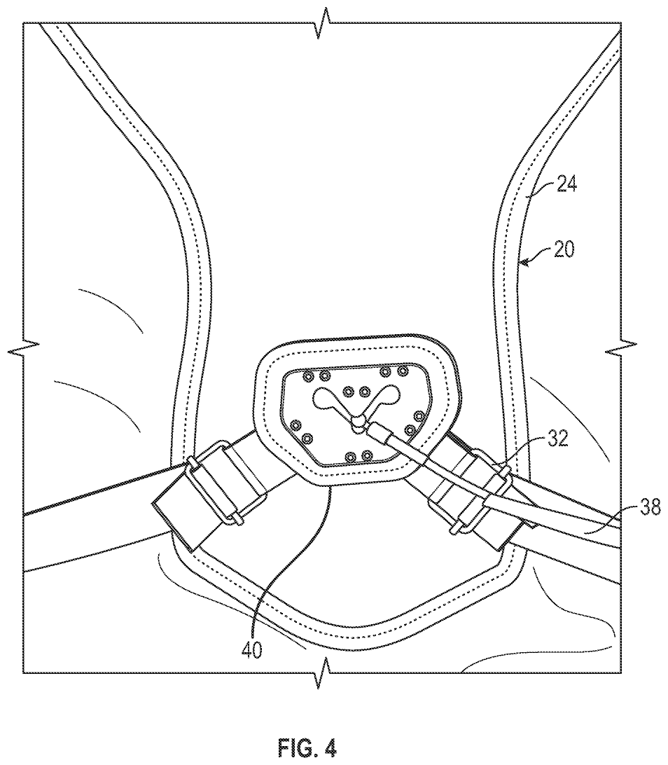

Therefore, an additional improvement and/or advantage of any of the aspects and/or embodiments disclosed herein includes the attachment of the prosthesis 34 to the harness 20 via the cable 38 for activation of the end effector 36. The connection may be made to the harness at a bracket 40 positioned at the rear portion 24 of the harness 20. According to at least some aspects of some embodiments, the bracket 40 is positioned to place pressure in the middle of the user's body. For example, the positioning of the bracket 40 on the rear portion 24 of the harness 20 may be such that the bracket 40 places pressure on or near the spine generally between the should blades of the user. Thus, the bracket 40 is designed to put pressure on or near the center of gravity of the bracket and user's body (see, e.g., FIGS. 3 and 4 for location of bracket).

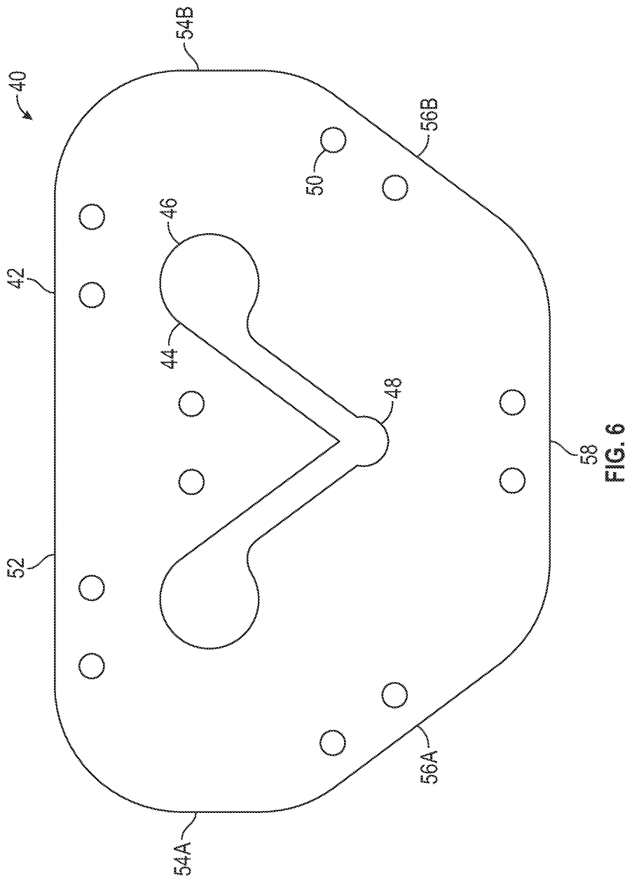

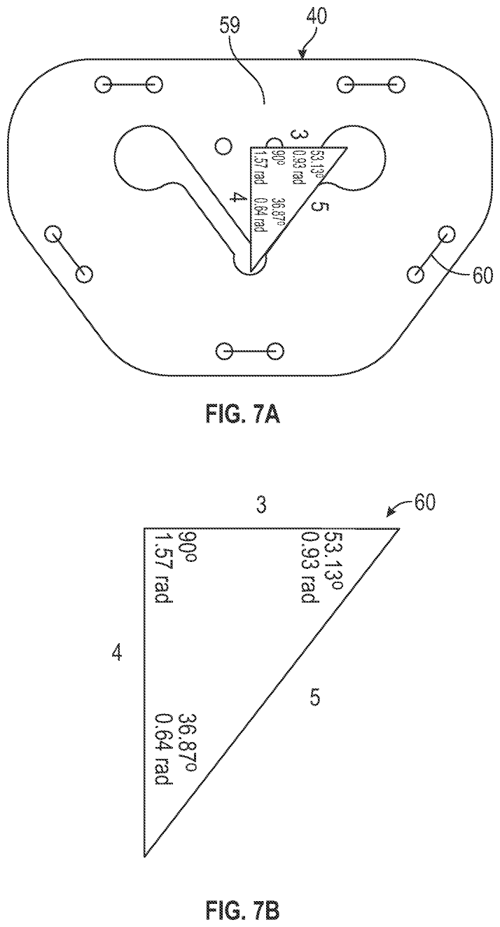

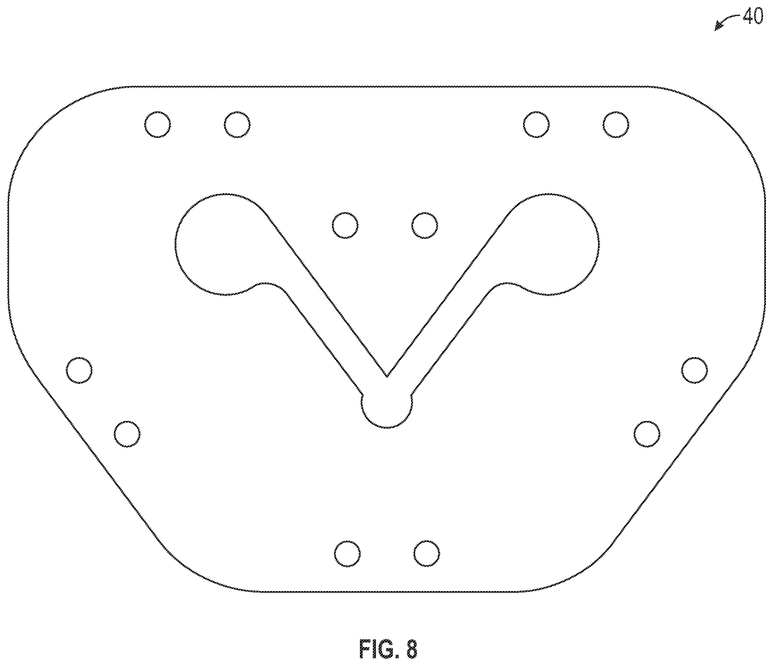

As shown best in FIGS. 6 and 8, the bracket 40 includes a bracket body 42 with opposite sides. The bracket 40 is shown to be an irregular hexagon shape with substantially parallel top and bottom sides 52, 58, as well as substantially parallel vertical sides 54A, 54B. The vertical sides angle inwards at angled sides 56A, 56B, such that the bottom 58 is generally narrower than the top 52. In addition, the bracket 40 is shown to be generally symmetrical about a bisecting line 59, which is shown in FIG. 7A.

While the bracket 40 is shown as a symmetrical, irregular hexagon shape, it should be appreciated that other shapes may be utilized. This can include, but is not limited to, triangles, quadrilaterals, ovals, or generally any polygon or shape that includes a bottom portion that is at least partially narrower than an upper portion, such as the top 52 and bottom 58 shown in the figures. Still further, it should be contemplated that the bracket 40 could be a type of channel shape, such as a half circle with two holes on either side.

As will be appreciated from the present disclosure, the narrower bottom portion as well as the symmetry provides numerous advantages. The bracket has a wider surface due to the wider top portion, which distributes the loads and pressure on the bracket and decreases the tension on the body. Because of the symmetry design in the bracket, it could be used for the left amputated side, right amputated side, or even for bi-lateral protheses needs.

Six double-holes 50 (spaced in configuration to the holes of a button) are placed on the bracket 40 through the bracket body 42 so that it can be firmly attached to the coordinating harness 20. This spacing and placement is used to secure the bracket into place.

Also shown extending through the bracket body are upwardly extending channels 48, which converge at or near a lower portion of the bracket body 42 and extend away from each other as they move towards the upper portion of the bracket body 42. According to at least some aspects and/or embodiments, the channels 44 form an upward V-shape from a common convergence location.

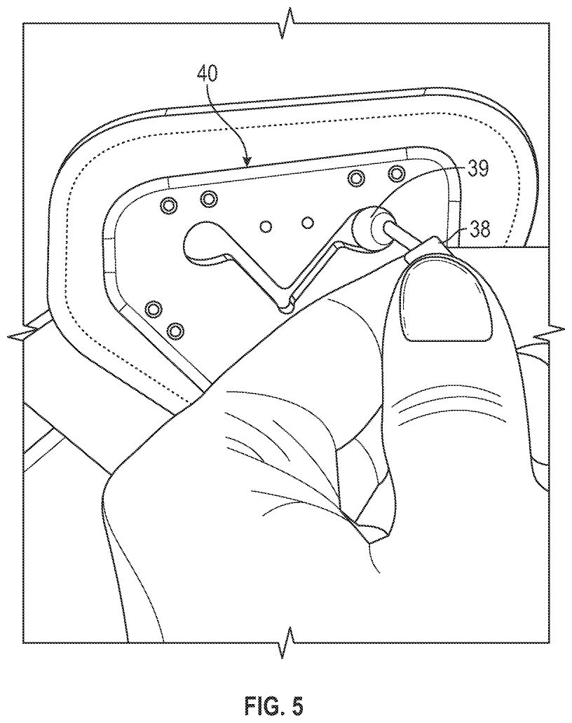

As noted herein, the cable 38 may include a ball 39 at one end (see, e.g., FIG. 5). This metal ball is the end of the standard cable used in body-powered upper limb prosthetic devices. Some metal balls 39 have one side that is curved with the other side being substantially flat with a spherical periphery. In such an instance, the flat side would be positioned towards the body of the user. The channels 38 include larger openings 46 at upper, distal ends away from the bottom or convergence point 48. The upper openings 46 allow the ball 39 of the cable to be attached to the bracket 40, which is attached to the harness 20, such as via sewing. In addition, the bracket 40 could be attached according to other means, including, but not limited to, PETG rivets or other types of rivets. FIG. 5 shows a depiction of the ball 39 being inserted in an opening 46. The ball 39 is then slid towards the convergence point 48, which is smaller than the diameter of the ball to hold the ball within the bracket for use of the prosthesis and harness. The ball 39 will be able to swivel and/or pivot within the channels 44, such as at the convergence point 48, to allow for full movement of the prosthesis and actuation thereof by the user.

As the bracket 40 is attached to the harness 20 and will be connected to the terminal device by the metal ball 39 (part of the prostheses cable unit), if users want to not use the prosthetic device, they can disconnect it from the harness and the harness can still remain on the body of the user. In other words, the bracket 40 could be used even when users choose to remove their prosthetic device(s) and there is no need to take it off (there is no hook or strict material in it to hurt the body or the user's clothing when they are not using the prosthetic device).

The harness design has potential for posture support because of the body shape and structure that supports chest/bust and spine, while being easily adjustable. This design also could be used for medical uses like placing magnets or TENS electrodes in it (such as along the spine or shoulders) for a kind of pain management or therapy.

While it is acknowledged that the channels 44 could take various angles for attaching the ball thereto, it has been found that the channels 44 work better when angled like a golden triangular 60 with 53-deg, 37-deg, and 90-deg. The golden triangle, sometimes also called the sublime triangle, is an isosceles triangle such that the ratio of the hypotenuse to base is equal to the golden ratio. For example, referring to FIGS. 7A and 7B, a triangle 60 taken along the bisecting ling 59 is shown. Based on these angles, the hook and fastener connected to the bracket and prosthetic device is placed in one direction and provides ease of movement.

According to at least some aspects and/or embodiments of the present disclosure, the bracket 40 comprises Polyethylene terephthalate glycol, commonly known as PETG. PETG is a thermoplastic polyester that provides significant chemical resistance, durability, and excellent formability for manufacturing. The PETG decreases the friction arising from traditional metal loops on previous harnesses.

The use of PETG includes numerous additional advantages. PETG is a less dense material compared to iron. The iron that was used in previous transradial prostheses is denser and heavier than plastic. As PETG is a thermoplastic polyester with ⅙ of the specific gravity compared to pure iron (1.325 g/cm3 to 7.870 g/cm3), it provides relatively lighter weight and more comfortability as carried by the body of the user.

PETG provides more sensorial comfort compared to Iron as it does not get cold or warm or attached to the body when it gets wet. PETG material was used for creating this bracket by 3D printing to decrease the friction arising from the metal loop on the back of the body. Unlike a metal loop, PETG will not stick/cling to the body nor to slipping on the body and making redness on the body skin and will not generate moisture leading to slipping on the body and making redness on the body. It also removes the feeling of cold from the metal loop and metal anchoring system. That means it mitigates feeling cold around the metal loop as it is alternated by PETG material. Likewise, there is less chance that the bracket will get hot, such as when exposed to the sun, unlike iron or other metals do when placed in direct sunlight.

Using PETG as a strong material based on the design which not using metal is preferable for still additional reasons. PETG provides significant chemical resistance, durability, and excellent formability for manufacturing, which has especially strong bonding capabilities between layers and is stronger than other options. Thus, it is a good choice for thin parts with high strength requirements. It is notable that the following properties in PETG are more than Iron; Stiffness to Weight: Bending, points (25 to 34), Strength to Weight: Axial, points (9.2 to 12), and Strength to Weight: bending, points (12 to 25).

PETG provides more body-friendly usage for a user because it could have a kind of bending and is not as sturdy as Iron. Thus, when amputees want to lean to the wall, it could be bent along the body shape. It is designed to be more body-friendly to not hurt the body when moving.

Finally, PETG is washable/launderable. This is beneficial as the iron rings could tend to rust if exposed to water or other liquids.

However, it is noted and should be appreciated that, while PETG is an advantageous material, other materials may be used based upon end-product performance expectations and production/retail costs.

For the embodiments shown, the PETG bracket was made via additive manufacturing, i.e., 3D printing. However, it should also be appreciated that the bracket could be formed by generally any known manufacturing process, including molding casting, or the like. The bracket 40 shown is a plate of 60*40 mm with 2 mm thickness.

### Example

To assess the functional performance of the harnessing system 10 as disclosed herein compared to a body-powered Figure-of-9 harness, two tests were conducted: NHPT (Nine-Hole Peg Test) and BBT (Box and Blocks Test). The objective of this study was to objectively assess and compare the functional performance of the new harness and the Figure-of-9 harness by using a TRS prosthesis simulator.

A convenience sample of 10 men and 10 women, all able-bodied individuals aged 18-52, participated in this study. All participants were right-handed and lacked experience in controlling a prosthesis. Due to the limited availability of upper limb amputees, the test opted for able-bodied subjects.

Before the test started, the test procedure was clearly explained to explained to the participants, and each person gave their informed consent.

For the Box and Blocks Test (BBT), participants were required to complete the test using the simulator equipped with the terminal device as quickly as possible. The trial concluded after 60 seconds. The BBT score was determined by the number of blocks transported within this 60-second period for each test.

The second test, the Nine-Hole Peg Test, required participants to pick up pegs one at a time using only the terminal device. They then placed the pegs into the holes in any order until all holes were filled. Following that, participants had to remove the pegs one at a time and return them to the container. The test duration was measured and expressed as seconds per peg movement (s/peg). There were a total of 18 peg movements: 9 placements and 9 removals. The test scores were expressed in s/peg by dividing the recorded time of a completed trial by the 18 peg displacements.

To ensure fairness in evaluating both harnesses, half of the participants commenced the evaluation with the Figure-of-9 harness, while the other half initiated the tests with the new harness. Prior to the tests, participants were given a 1-minute practice period for both tests. It is essential to note that participants wore both harnesses over their clothing.

The results, illustrated in the following table, indicate a noteworthy improvement in performance with the new harness. Ninety-five percent of participants demonstrated superior performance in the Box and Blocks Test, while an 85% improvement in total time for placement and displacement was observed in the Nine-Hole Peg Test compared to the Figure of 9 harness.

TABLE 1
Nine Hole Peg Test Results

| | | | Figure of | New | |
|---|---|---|---|---|---|
| | Figure of | New | 9 Harness | harness | Performance |
| Par- | 9 Harness | harness | s/peg for | s/peg for | improvement |
| tici- | Total Time | Total | Figure of | new | between two |
| pants | (s) | Time (s) | 9 Harness | harness | harnesses |
| P1 | 105 | 102 | 5.83 | 5.67 | 0.17 |
| P2 | 114 | 102 | 6.33 | 5.67 | 0.67 |
| P3 | 176 | 129 | 9.78 | 7.17 | 2.61 |
| P4 | 72 | 55 | 4 | 3.06 | 0.94 |
| P5 | 140 | 89 | 7.78 | 4.94 | 2.83 |
| P6 | 96 | 74 | 5.33 | 4.11 | 1.22 |
| P7 | 100 | 95 | 5.56 | 5.28 | 0.28 |
| P8 | 95 | 78 | 5.28 | 4.33 | 0.94 |
| P9 | 99 | 71 | 5.5 | 3.94 | 1.56 |
| P10 | 149 | 106 | 8.28 | 5.89 | 2.39 |
| P11 | 90 | 65 | 5 | 3.61 | 1.39 |
| P12 | 100 | 90 | 5.56 | 5 | 0.56 |
| P13 | 70 | 89 | 3.89 | 4.94 | −1.06 |
| P14 | 120 | 114 | 6.67 | 6.33 | 0.33 |
| P15 | 80 | 54 | 4.44 | 3 | 1.44 |
| P16 | 170 | 119 | 9.44 | 6.61 | 2.83 |
| P17 | 82 | 71 | 4.56 | 3.94 | 0.61 |
| P18 | 102 | 107 | 5.67 | 5.94 | −0.28 |
| P19 | 147 | 108 | 8.17 | 6 | 2.17 |
| P20 | 87 | 84 | 4.83 | 4.67 | 0.17 |

TABLE 2
Box and Block Test Results

| | Figure of | New | | | |
|---|---|---|---|---|---|
| | 9 Harness | harness | | | |
| | Number of | Number of | | | |
| | Cube in | Cube in | Figure of | New | Performance |
| Par- | Box and | Box and | 9 Harness | harness | improvement |
| tici- | Blocks Test | Blocks Test | Time per | Time per | between two |
| pants | in 60 s | in 60 s | cube | cube | harnesses |
| P1 | 11 | 18 | 5.45 | 3.33 | 2.12 |
| P2 | 9 | 18 | 6.67 | 3.33 | 3.33 |
| P3 | 16 | 19 | 3.75 | 3.16 | 0.59 |
| P4 | 26 | 28 | 2.31 | 2.14 | 0.16 |
| P5 | 22 | 24 | 2.73 | 2.5 | 0.23 |
| P6 | 12 | 17 | 5 | 3.53 | 1.47 |
| P7 | 18 | 19 | 3.33 | 3.16 | 0.18 |
| P8 | 19 | 24 | 3.16 | 2.5 | 0.66 |
| P9 | 18 | 20 | 3.33 | 3 | 0.33 |
| P10 | 23 | 26 | 2.61 | 2.31 | 0.3 |
| P11 | 22 | 26 | 2.73 | 2.31 | 0.42 |
| P12 | 24 | 24 | 2.5 | 2.5 | 0 |
| P13 | 18 | 20 | 3.33 | 3 | 0.33 |
| P14 | 9 | 13 | 6.67 | 4.62 | 2.05 |
| P15 | 20 | 21 | 3 | 2.86 | 0.14 |
| P16 | 13 | 17 | 4.62 | 3.53 | 1.09 |
| P17 | 20 | 25 | 3 | 2.4 | 0.6 |
| P18 | 21 | 22 | 2.86 | 2.73 | 0.13 |
| P19 | 15 | 18 | 4 | 3.33 | 0.67 |
| P20 | 18 | 22 | 3.33 | 2.73 | 0.61 |

These findings suggest that the innovative bracket and harnessing system have the potential to enhance upper-limb body-powered prosthetic device users' performance by 85% and 95% in the respective functional assessment tests.

Based on participant feedback, the consensus is that the harnessing system 10 of the present disclosure is perceived as more comfortable, secure, and easier to control. Participants highlighted that it exerts less pressure on the unamputated side of the body and allows for a broader range of motion. Some participants specifically noted improved ease of movement for pulling the hand from various angles and a more comfortable weight distribution.

Therefore, an improved harness system, harness, and attachment bracket for use with a prosthesis has been shown and/or described. It should be appreciated that variations, alternatives, or other changes obvious to those skilled in the art should be considered a part of the present disclosure. In addition, it should be appreciated that any portion, component, and/or aspect of any embodiment shown, described, or otherwise suggested could be combined in a manner that includes embodiments not explicitly disclosed, but which are to be considered a part of the present disclosure.

1. A harness system, comprising:
a harness comprising front and rear portions connected by an open upper area, said front and rear portions extending generally downward at central locations;
a bracket positioned at the rear portion of the harness, said bracket comprising upwardly V-shaped channels;
wherein said bracket positioned on the rear portion of the harness in a manner to position the bracket at a generally center portion of the rear portion.

2. The harness system of claim 1, further comprising a prosthetic device operatively connected to the bracket.

3. The harness system of claim 2, wherein the prosthetic device comprises a cable attached to one or both of the upwardly V-shaped channels of the bracket.

4. The harness system of claim 3, wherein the prosthetic device further comprises an end effector actuated via the cable.

5. The harness system of claim 1, wherein the bracket comprises polyethylene terephthalate glycol (PETG).

6. The harness system of claim 1, wherein the bracket comprises an irregular hexagon shape.

7. The harness system of claim 6, wherein the bracket is symmetrical.

8. The harness system of claim 1, wherein the upwardly V-shaped channels comprise larger openings at the upper portions of the channels.

9. The harness system of claim 1, wherein the upwardly V-shaped channels are in a golden triangular configuration.

10. The harness system of claim 1, wherein the harness further comprises straps extending from opposite sides of the front and rear portions at lower portions thereof.

11. A bracket for use with a harness system including a prosthetic device, comprising:
a bracket body;
upwardly V-shaped channels extending through the bracket body, said channels being symmetrical and angled to form a golden triangle;
wherein said bracket comprising PETG.

12. The bracket of claim 11, wherein the bracket body is in an irregular hexagon shape.

13. The bracket of claim 12, wherein the bracket body is symmetrical.

14. The bracket of claim 11, wherein the upwardly V-shaped channels comprise larger openings at the upper portions of the channels.

15. The bracket of claim 11, further comprising a plurality of apertures positioned around the periphery of the bracket body for attachment to a harness.

16. The bracket of claim 15, further comprising rivets positioned at the plurality of apertures to attach the bracket body to the harness.

17. A harness for use with a prosthetic device, comprising:
a harness body comprising front and rear portions connected by an open upper area, said front and rear portions extending generally downward at central locations;
a bracket positioned at the rear portion of the harness, said bracket comprising upwardly V-shaped channels; and
straps extending from opposite sides of the front and rear portions at lower portions thereof;
wherein said bracket positioned on the rear portion of the harness in a manner to position the bracket at a generally center portion of the rear portion.

18. The harness of claim 17, wherein said bracket comprising a symmetrical hexagon shape with inwardly oriented lower sections.

19. The harness of claim 18, wherein said bracket comprises a plurality of attachment apertures for attaching the bracket to the harness body.

20. The harness of claim 17, wherein the upwardly V-shaped channels comprise larger openings at the upper portions of the channels.
